(19) United States
(12) Patent Application Publication
(10) Pub. No.: US 2013/0102829 A1
(43) Pub. Date: Apr. 25, 2013

(54) METHOD FOR TREATING OSTEOSARCOMA BY BORON NEUTRON CAPTURE THERAPY USING BORIC ACID AS A BORON DRUG

(71) Applicants: Fong-In CHOU, Hsinchu City (TW); Chen-Fang SHU, Changhua County (TW); Jinn-Jer PEIR, Hsinchu City (TW); Hong-Ming LIU, Hsinchu City (TW); Jiunn-Wang LIAO, Taichung (TW); Yung-Chang LIN, Taichung (TW)

(72) Inventors: Fong-In CHOU, Hsinchu City (TW); Chen-Fang SHU, Changhua County (TW); Jinn-Jer PEIR, Hsinchu City (TW); Hong-Ming LIU, Hsinchu City (TW); Jiunn-Wang LIAO, Taichung (TW); Yung-Chang LIN, Taichung (TW)

(73) Assignee: National Tsing Hua University, Hsinchu (TW)

(21) Appl. No.: 13/280,466

(22) Filed: Oct. 25, 2011

(51) Int. Cl. A61N 5/00

(52) U.S. Cl. 600/1

(57) ABSTRACT

The present invention provides a treatment of osteosarcoma by boron neutron capture therapy using boric acid as a single boron drug.

### FIELD OF THE INVENTION

The present invention is related to a treatment of osteosarcoma by boron neutron capture therapy (BNCT), and in particular to a treatment of osteosarcoma by BNCT using boric acid as a single boron drug.

### BACKGROUND OF THE INVENTION

Although adjuvant chemotherapy with extensive surgery treatment is standardized use for osteosarcoma treatment in clinic, however, the prognosis is poor with frequent pulmonary metastasis. Osteosarcoma is radioresistant to standard dose of radiotherapy, so radiotherapy is not used for treating this tumor type. However, while high dose irradiation may be administered after extensive surgery or the risk of metastases of osteosarcoma, postoperative radiotherapy wounds normal tissues. BNCT provides a way to selectively destroy malignant cells and spare normal cells. It is based on a sufficient amount of ¹⁰B which must be selectively delivered to the tumor, or tumor vasculature, and enough thermal neutrons must be absorbed by them. The nuclear reaction of ¹⁰B(n, α)⁷Li yields high linear energy transfer (LET) a particles and recoiling ⁷Li. These densely ionizing particles can release their energies within a short range (about 9 μm for α and 5 μm for ⁷Li, respectively) and result in effective destructions of tumor cells.

The clinical trials using BNCT had included boron drugs such as: sodium borate, borocaptate sodium, and boronophenylalanine. Sodium borate (Borax) was used as the boron drug in the BNCT clinical trials for treating brain tumors in the 1950s, but the trials were unsuccessful and thus Borax was not used in the BNCT clinical trials later. Borocaptate sodium (BSH) and boronophenylalanine (BPA) have both been approved by the FDA to be used as the boron drugs in the BNCT clinical trials. BSH is a water-soluble boron drug commonly used in the BNCT clinical trials for treating Glioblastoma multiforme patients, but the injection of BSH does not result in higher ratio of boron concentrations between that from tumors and normal tissues. BPA is a derivative of phenylalanine, and when treating melanoma or head and neck cancers, the injection of BPA yields higher accumulation of BPA in tumor tissues than that in normal tissues. However, the accumulation of BPA in tumors is ineffective when treating osteosarcoma. [Ferrari, C., Zonta, C., Cansolino, L., Clerici, A. M., Gaspari, A., Altieri, S., Bortolussi, S., Stella, S., Bruschi, P., Dionigi, P., Zonta, A., “Selective uptake of p-boronophenylalanine by osteosarcoma cells for boron neutron capture therapy,” Applied Radiation and Isotopes, vol. 67, pp. 5341-344, 2009]. Therefore there is still an outstanding need for an effective boron drug for BNCT for osteosarcoma.

### SUMMARY OF THE INVENTION

A primary objective of the present invention is to provide a novel boron drug for boron neutron capture therapy (BNCT) for osteosarcoma.

Another objective of the present invention is to provide a method for treating osteosarcoma by BNCT.

In order to accomplish the aforesaid objectives a novel boron drug for BNCT for osteosarcoma provided by the present invention is boric acid.

A method for treating osteosarcoma by BNCT provided by the present invention comprises administering boric acid to a subject suffering osteosarcoma as a boron drug for BNCT, and subjecting the osteosarcoma in the subject to neutron irradiation.

Preferably, the boric acid administered is an aqueous solution. More preferably, the administering comprises intravenously injecting the aqueous solution of boric acid to the subject.

Preferably, the boric acid is constituted by boron having a major portion, and more preferably about 99%, of ¹⁰B stable isotope.

Preferably, said subject is human.

Preferably, the boric acid is administered in an amount of 5-60 mg of ¹⁰B stable isotope per kilogram of human body weight. The previous study shows that an injection dose of boric acid with 60 mg of ¹⁰B stable isotope per kilogram of human body weight has no permanent adverse effect. Accordingly, the boric acid is administered with an amount not exceeding 60 mg of ¹⁰B stable isotope per kilogram of human body weight to avoid adverse effects, unless future study indicates otherwise.

Preferably, the method of the present invention further comprises measuring a concentration of ¹⁰B stable isotope in the subject's blood, wherein said neutron irradiation is carried out according to the measured concentration.

Preferably, said neutron irradiation is carried out when the measured concentration is higher than 20 μg ¹⁰B/g. Said neutron irradiation is carried out generally about 60-180 minutes after the intravenous injection.

Preferably, said neutron irradiation is carried out with a physical dose of 5-15 Gy, and more preferably 7-12 Gy.

The method of the present invention has at least the following advantages:
1. The method of the invention allows the drugs to be injected intravenously, and the affected part of a patient does not need to be extracted by surgery and irradiated ex vivo; a tumor is subjected to neutron irradiation in a patient's body without surgery.
2. Because osteosarcoma is characterized by the formation of bone or osteoid by the tumor cells, while boric acid yields higher specific accumulation in the bone or osteoid in the tumor tissues, but not in the soft tissues of a living creature, the boron drug of the invention effectively results in differences in the boron concentrations of the bones and the soft tissues. In addition, the vasculature of a tumor is different from that of a normal tissue, so when an osteosarcoma undergoes neutron irradiation, the vasculature (blood vessels supplying nutrients to the tumor) suffers more damage than that of the normal tissue.
3. When an osteosarcoma undergoes neutron irradiation, because there are no critical organs present around the femur, it is possible to give a patient a higher physical dose of BNCT irradiation once or multiple times in the evaluation of physical doses, so as to kill tumor cells effectively.
4. Due to the fact that boric acid does not show specific accumulation in the soft tissues of a living creature, the boron concentration in a patient's blood is the approximate boron concentration in his/her soft tissues. Therefore, the blood boron concentration and the maximum allowable dose for the normal tissues surrounding a tumor can be used to calculate the dose required for BNCT irradiation. As a result, Positron Emission Tomography (PET) is not required to estimate the ratio of boron concentrations between the blood (or normal tissues) and the tumor before carrying out neutron irradiation.

### BRIEF DESCRIPTION OF THE DRAWINGS

FIG. 1 shows the boron concentrations in major organs, tumor tissue and bones in osteosarcoma-bearing rats. The rats were tail vein injection of boric acid at a dose of 25 mg ¹⁰B/kg body weight. The bron concentration of the major organs, tumor and various bone tissues were assayed at 1^{st}, 2^{nd} and 3^{rd} hour after boric acid injection. Each bar represents the mean±SD (n=3).

FIG. 2 shows the body weight of the osteosarcoma-bearing rats within 80 days after the BNCT treatment. In tumor control group, the osteosarcoma-bearing rats did not receive BNCT treatment; in the high dose

BNCT group, tumor regions of the osteosarcoma-bearing rats were received a dose of 11.0 Gy; and in the low dose BNCT group, tumor regions of the osteosarcoma-bearing rats were received a dose of 5.8 Gy. Each point represents the mean±SD (n=3-5). In tumor control group, the rats were sacrificed after which the tumor was too large, and data were collected until 41 days after implantation of tumor cells (which are equal to the 30^{th} day after BNCT of the treated groups).

FIG. 3 shows the relative tumor area before and after BNCT treatment by tracing the tumor region based on radiography in osteosarcoma-bearing rats within 80 days after BNCT. In tumor control group, the osteosarcoma-bearing rats did not receive BNCT treatment; in the high dose BNCT group, tumor regions of the osteosarcoma-bearing rats were received a dose of 11.0 Gy; and in the low dose BNCT group, tumor regions of the osteosarcoma-bearing rats were received a dose of 5.8 Gy. The tumor area of rat in each group was compared with that of the same rat on the day of BNCT treatment. Each bar represents the mean±SD (n=3-5, **p<0.01 compared with controls, *p<0.05 compared with controls). In tumor control group, the rats were sacrificed and data were collected until day 30.

FIG. 4 shows radiographs taken for the same rats on 1 day before BNCT and 30, 60, 80 days after BNCT treatment for the tumor control group, the high dose BNCT group, and the low dose BNCT group, respectively. In tumor control group, the rats were sacrificed and data were collected until day 30, after which the tumor was too large to be accurately measured.

FIG. 5 The radiographs show the hind limbs of osteosarcoma-bearing rats with and without BNCT treatment. (A1) and (A2) are radiographs taken for the same rat on the 5^{th} and 16^{th} day after the high dose of BNCT treatment, respectively. These show the bone healing process from the 5^{th} to 16^{th} day in the high dose BNCT group. (A1) shows the osteolysis lesion within the distal anterior femur, and the radiolucent line (arrow) in the femoral metaphysis just on the 5^{th} day after BNCT. (A2) shows the increased radiodensities (arrow) at the site of bone healing on the 16^{th} day after BNCT. (B1) and (B2) are radiographs taken for the same rat on the 5^{th} and 23^{th} day after the low dose of BNCT treatment, respectively. These show the bone healing process from the 5^{th} to 23^{th} day in the low dose BNCT group. (B1) shows the osteolytic lesion within the cortical bone of distal posterior femur (arrow) just on the 5^{th} day after BNCT. (B2) shows the callus formation on the 23^{th} day after BNCT, which became continuity through fracture line (arrow). (C1) and (C2) are radiographs taken for the same rat on the 16^{th} and 41^{th} day after implantation of tumor cells (which are equal to the 5^{th} and 30^{th} day after BNCT of the treated groups) showing the tumor conditions in the control group. (C1) shows the osteolysis at the distal posterior femur (arrow) and the elevation of periosteum on the 16^{th} day after implantation of tumor cells. (C2) shows full-thickness bicortical bone loss, and progressive radiolucent lesion (arrow) on the 41^{th} day after implantation of tumor cells.

FIG. 6 shows the histopathological finding of the extra-skeletal regions of osteosarcoma with hematoxylin and eosin stain in osteosarcoma-bearing rat on 80^{th} day after BNCT. (A) The histological section of the scar tissue resulting from the extra-skeletal regions of tumor after BNCT. (B) This histological section from the high magnification of the field indicated in (A). The mass of scar tissues observed on radiographs were osteoid (OST) and fibrous connective tissues (FCT). The osteoid was present in the left darker area, fibrous connective tissue and multinucleated giant cell (MGC) were present in the right ocean grey area, No survival tumor cells were found in histological examination. (CB: Cortical Bone; MU: Muscle; BM: Bone marrow).

### DETAILED DESCRIPTION OF THE INVENTION

In vivo and in vitro studies indicate that boric acid (B(OH)₃) (BA) has a strong affinity with hydroxyapatite of bone owing to the binding to the cis-hydroxy groups. It has been proved that the ratio of boron concentrations in blood and soft tissue is approximately equal to one [Chou, F. I., Chung, H. P., Liu, H. M., Chi, C. W., Lui, W. Y., “Suitability of boron carriers for BNCT: accumulation of boron in malignant and normal liver cells after treatment with BPA, BSH and BA,” Applied Radiation and Isotopes vol. 67, pp. 105-108, 2009.]. Murry, F. J. discovers that the boron concentrations in bones exceeded those in blood by a factor of 4 through an oral administration of BA to rats or human [Murry, F. J., “A comparative review of the pharmacokinetics of boric acid in rodents and human,” Biological Trace Element Research, vol 66, pp. 331-341, 1998.]. Boron accumulation is characterized by its involvement of bone remodeling [Eckhert, C., Barranco, W., Kim, D., “Boron and prostate cancer a model for understanding boron biology,” In: Xu, F., Goldbach, H. E., Brown, P. H., Bell, R. W., Fujuwara, T., Hunt, C. D., Goldberg, S., Shi, L. (Eds.), Advances in Plant and Animal Boron Nutrition, Springer, Dordrecht, pp. 291-297, 2007.]. The inventors of the present application are the first to conceive an idea of using boric acid as a single boron drug in treating osteosarcoma by BNCT.

In the following experiments the inventors of the present application will prove that a situation in which BA accumulates in osteosarcoma due to high boron retention in bone and the bone formation of cancer cells makes BNCT a promising therapy for osteosarcoma by using BA as the sole boron drug for BNCT. The experiments are merely for illustrative, and not for limiting a scope of the present invention.

### EXPERIMENTS

### Materials and Method

1. The Preparation of a Boron Drug and the Measurement of Boron Concentration Thereof.

According to the invention, the boron drug used in BNCT for treating osteosarcoma is boric acid.

The Preparation of Boric Acid Aqueous Solution

The chemical formula for boric acid is H₃BO₃, which has a molecular weight of 61.83. The boron atom of boric acid used in the experiment comprises 99% of ¹⁰B boron atoms, and 1% of ¹¹B boron atoms (Sigma-Aldrich Co. LLC). The boric acid aqueous solution was prepared by adding an adequate amount of boric acid powder into a saline solution to attain the required ¹⁰B concentration.

The Measurement of Boron Concentration

Before measuring boron concentration of a sample, it needs to undergo microwave digestion first. A sample was initially placed into a Teflon high-pressure digestion vessel, then added with 3 mL of concentrated nitric acid (14N, 65%) and 0.5 mL of hydrogen peroxide (30-35%), and the vessel was sealed before placing the vessel into a microwave digestion system (MLS 1200 Milestone, Italia) for sample decomposition. The decomposition was achieved in two stages; the first stage of digestion proceeded for 3 minutes with power set at 300 W, and the second stage of digestion proceeded for 2 minutes with power set at 600 W. Finally, the sample was allowed to cool and depressurize for 20 minutes. The sample subsequently turned into a clear solution after complete digestion. After diluting the sample with deionized water, an Inductively Coupled Plasma Atomic Emission Spectrometer (ICP-AES, OPTIMA 2000 DV, PerkinElmer Instruments) was used to measure the boron concentration of the sample. For the conditions of the analysis, the temperature of argon plasma was set at 6000-7000 K; the analytical wavelength was set at 249.773 nm, and the liquid uptake rate was set at approximately 2 mL/min; the operating voltage after the formation of plasma was set at 40 V.

2. The Establishment of Osteosarcoma-Bearing Animal Model

The osteosarcoma-bearing animal model was established by using the Sprague-Dawley (SD) rats, so as to carry out animal experiments using a neutron capture therapy to treat osteosarcoma.

The Implantation of Tumors

Male SD rats were the animals used in this osteosarcoma-bearing animal-model. Prior to the experiments, the rats were bred in cage for at least one week at temperature of 22° C. and humidity of 30-70%. An artificial lighting condition (12 h on/12 h off) was maintained in the room. The animal feeds and water were unlimited. The tumor cells used in the experiments were UMR-106 cell strain (Rat osteogenic sarcoma cell line, BCRC No: 60270). Firstly, obtained the UMR-106 tumor cells in exponential growth phase from a culture flask, then centrifuged the UMR-106 cells obtained by subculture method, followed by re-suspending the cells in a media without serum or antibiotics, and dividing the suspension into separate centrifuge microtubes. Placed the Matrigel Matrix (BD Bioscience), a syringe, and the centrifuge microtubes with the cell suspension on ice to be cooled for later use. Before implanting the tumor cells, the UMR-106 cell suspension was mixed with an equal amount of Matrigel first.

A SD rat was firstly anesthetized by using 2.5% Isoflurane (Halocarbon Laboratories, River Edge, N.J.), followed by shaving and disinfecting a surgical area on the rat for implanting the tumor cells. A 250 μL micro-syringe was fitted with a 25 gauge needle before extracting 200 μL of a mixed solution containing the cells and Matrigel (10×10⁶ cells/200 μL). Then the needle was used to penetrate a SD rat's distal femur, which is the femoral condyle; the syringe was rotated in order to penetrate the femur and the spongy bone, and ensured it had protruded out of the femur by touching with a finger. Subsequently, 100 μL of the tumor cell solution was slowly injected into the extra-skeletal muscular tissues, and 100 μL of the solution was injected into the epiphysis and the metaphysis of the femur (at 0.5 cm deep from skin surface) after slowly retracting the needle. After the implantation, the rats were released back into the cage, and their activities and body conditions were monitored continuously. The animals were immunosuppressed by daily administration of cyclosporin A (10 mg/kg, Sandimmun®, Novartis) for total 7 days from the 3^{rd} to the 9^{th} day after UMR-106 cells injection.

3. The Measurement of Boron Drug Distribution in the Rats

Using the boric acid aqueous solution as a boron drug to do the animal experiments, drugs were administered to the osteosarcoma-bearing rats (six-week old, approximately 200-250 g in body weight) via tail vein injection, and the dose of the boric acid drugs was: 25 mg ¹⁰B/kg BW. The rats' body weights needed to be measured and recorded before the experiments for preparing the boric acid aqueous solution.

The Measurement of Tissue Boron Concentration After Administering the Boron Drug to the Rats

After tail vein injection of boric acid, the rats were sacrificed at 1st, 2nd and 3rd hours after the injection. The rats' hearts, livers, stomachs, pancreas, spleens, lungs, kidneys, muscles, tumors, blood, testicles, proximal femurs bearing tumor, distal femurs bearing tumor, normal proximal femurs, normal distal femurs, tibias, lumbar vertebrae, humeri, sterna, ribs, and intestines were extracted and frozen at −20° C. for analyses of tissue boron concentrations later.

4. Boron Neutron Capture Therapy (BNCT)

The Design of Neutron Irradiation Fixture for Irradiating

Osteosarcoma-bearing Rats and Calculation of Dose Thereof The collimator to be used for neutron irradiation on osteosarcoma-bearing animals was for irradiating tumors on the-rats' legs, and was made of three fixing frames extending outwards; it allowed three rats to be irradiated simultaneously. The fixing frames consisted of polymethylmethacrylate (PMMA) and polyethylene (PE). Firstly, pulled a leg of the rat out of a securing device and toward a center of a beam aperture. Subsequently, the Monte Carlo N-Particle transport code system (MCNP) and a source file thereof were used to simulate and calculate the flux of neutrons from an outlet of the beam aperture through different materials and thicknesses on a living creature, so as to optimize conditions for the irradiation. For a reference about the parameters of the elemental composition of different materials and the rats, the material composition report from NIST (National Institute of Standards and Technology) and the 46^{th} report from ICRU (International Commission on Radiation Units & Measurements) were referred, and volumes and positions of the organs were specified by using mathematical formulas according to the sizes of the real organs. Finally, the dose rates for the organs of the rats were simulated by using the MCNP application, so as to determine a treatment dose and an irradiation time for each of the rats.

The Establishment of Neutron Irradiation Modes for Irradiating Osteosarcoma in Animals

The experimental animals were divided into three groups; a tumor control group (in which the rats received implantation of tumor cells but not BNCT treatment), a high-dose BNCT group, and a low-dose BNCT group.

After the SD rats were implanted with tumor cells for 11 days, the BNCT treatment and subsequent evaluation of biological effects were commenced. Considering the pharmacokinetics and the changes in boron concentration distribution in the rats after the drug administration, BNCT irradiation was carried out at adequate times after administering boric acid. The rats were anesthetized by gas, then intravenously injected with a boric acid of 25 mg ¹⁰B/kg BW. Fifteen minutes before the neutron irradiation, the rats were injected with Atropine and Zoletil to allow the rats to be anesthetized longer. Each of the rats was placed on a fixing frame with its tumor-bearing area close to the beam aperture of the Tsing Hua Open-pool Reactor (THOR), and then neutron irradiation was performed at a specific neutron flux and irradiation time to achieve a certain total neutron fluence. For the reactor power at 1.2 MW, the neutron fluxes at the central beam exit were 1.34×10⁸, 1.07×10⁹, and 7.66×10⁷ n/cm²/sec, respectively for thermal, epithermal, and fast neutrons. [Liu, Y. H., Tsai, P. E., Liu, H. M., Jiang, S. H., “Characterization of a BNCT beam using neutron activation and indirect neutron radiography,” Radiation Measurements, vol. 45, pp. 1167-1170, 2010.] After the irradiation, the actual irradiation time and reactor power were adjusted by using the on-line neutron monitoring system for THOR.

5. The Statistical Analyses

The values of the experimental results are shown as Mean±SD (Standard Deviation). The data of the treatment groups and the tumor control group were compared by Student's t-test, in which a p-value less than 0.05 indicated the presence of significant differences.

### Results

1. The Distribution of Boric Acid in a Living Creature

The Biodistribution of the Boron Drug in an Osteosarcoma-Bearing SD Rat.

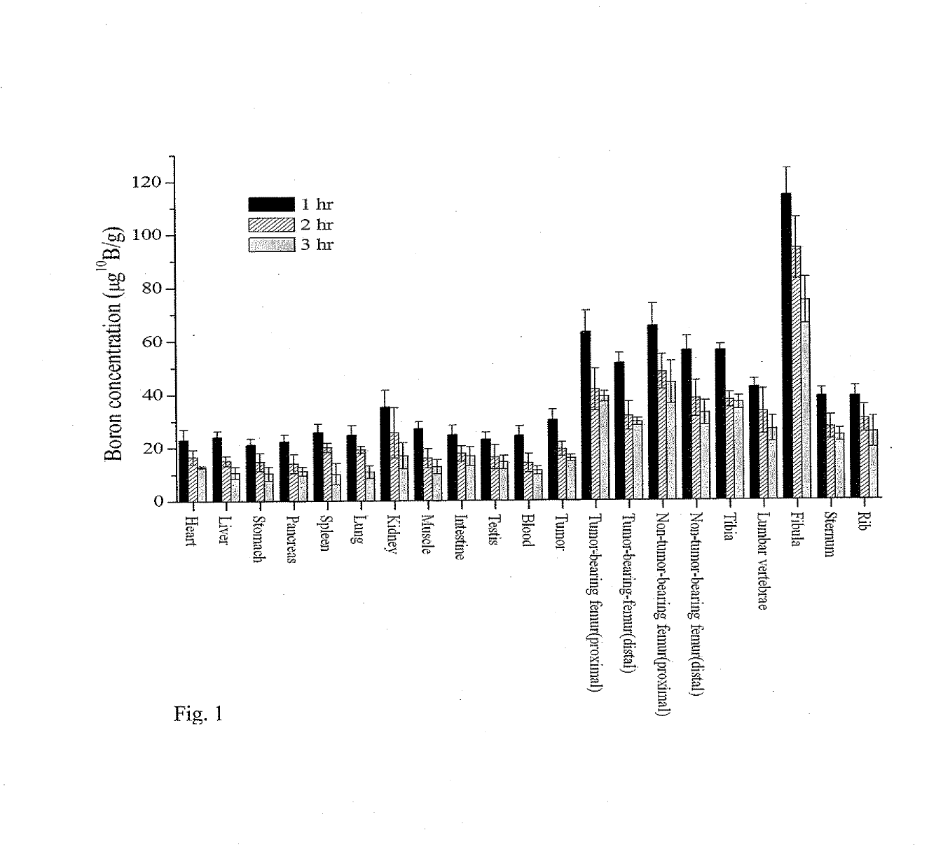

On the 11th day after the tumor implantation, the osteosarcoma-bearing rats were intravenously injected with a boric acid (25 mg ¹⁰B/kg BW) thereof, then their organs, bones and tumors were extracted for analyzing boron concentrations therein at the 1^{st}, 2^{nd} and 3^{rd} hours after the injection. FIG. 1 shows the boron concentrations in the major organs, tumor tissues and bones in osteosarcoma-bearing rats that had tail vein injection of boric acid; at the 1^{st}, 2^{nd} and 3^{rd} hour after the injection. Boron concentrations in the soft tissues and blood of the rats were similar. At one hour after the injection, the boron concentrations in the rats' blood, extra-skeletal tumors, tumor-bearing proximal femurs, tumor-bearing distal femurs, normal proximal femurs, normal distal femurs, tibias, lumbar vertebrae, fibulas, sterna, and ribs were 24.3±3.5, 30.3±3.9, 63.2±8.2, 51.6±3.7, 65.4±8.2, 56.2±5.4, 56.3±2.2, 42.5+3.0, 114.5±10.0, 38.9±3.2, and 39.0±3.9 μg ¹⁰B/g, respectively. At two hours after the injection, the boron concentrations of the organs mentioned above were 14.1±3.4, 19.3±2.7, 41.5±7.8, 31.7±5.4, 48.2±6.5, 38.2±6.7, 37.6±2.86, 33.2±8.5, 94.5±11.6, 27.3±4.5, and 30.4±5.3 μg ¹⁰B/g, respectively. At three hours after the injection, the boron concentrations of the organs mentioned above were 11.2±1.5, 15.8±1.3, 39.0±1.9, 29.5±1.4, 44.2±7.9, 32.8±4.6, 36.7±2.5, 26.7±4.8, 74.7±7.7, 24.4±2.5, and 25.4±4.8 μg ¹⁰B/g, respectively. At one hour after the injection, the extra-skeletal tumor tissues had a boron concentration approximately 1.2 times higher than that in the blood; the tumor-bearing proximal femurs had a boron concentration approximately 2.6 times higher than that in the blood, whereas the tumor-bearing distal femurs had a boron concentration approximately 2.1 times higher than that in the blood.

2. The Physical Dose at Each of the Positions and Organs

The osteosarcoma-bearing rats underwent BNCT treatment at the 11^{th} day after the tumor implantation, and the average diameter of the tumors before the treatment was 2.0±0.5 cm, but degrees of calcification in the tumors before the treatment were unclear. Boric acid (25 mg 10B/kg) were injected into the tumor-bearing rats via the tail vein before the irradiation. Afterwards, the rats were anesthetized at 15 minutes before the irradiation, and then placed on the fixing frames. The THOR beam used for the irradiation was epithermal neutron beams, and the reactor was allowed to operate at 1.5 MW; the low-dose and high-dose BNCT groups were designated with neutron irradiation times of 30 and 60 minutes, respectively. After the irradiation, the on-line neutron monitoring system for THOR was used to obtain the actual irradiation times and powers. Consequently, the doses received in each of the organs were calculated according to the actual irradiation times and powers, along with simulations of dose rates in the rats' organs from the MCNP application. Table 1 shows the doses received in the osteosarcoma-bearing SD rats' tumor tissues and organs near the tumors after the irradiation, from the low-dose and high-dose BNCT groups.

TABLE 1
The physical doses and dose component in the osteosarcoma-bearing
rats’ tumor tissues and organs near the tumors in the low-dose and high-dose
BNCT groups

| | Low dose BNCT group (Gy) | | | | High dose BNCT group (Gy) | | | |
|---|---|---|---|---|---|---|---|---|
| | (% of total dose) | | | | (% of total dose) | | | |
| | Total | | | | Total | | | |
| Organ | dose | Neutron (%) | Gamma (%) | Boron (%) | dose | Neutron (%) | Gamma (%) | Boron (%) |
| Rat body | 1.3 | 0.1 | 0.4 | 0.8 | 2.5 | 0.3 | 0.7 | 1.5 |
| | | (7.7%) | (30.8%) | (61.5%) | | (12.0%) | (28.0%) | (60.0%) |
| Femur | 7.9 | 0.6 | 1.0 | 6.3 | 15.1 | 1.1 | 2.0 | 12.0 |
| | | (7.6%) | (12.7%) | (79.7%) | | (7.3%) | (13.2%) | (79.5%) |
| Tumor | 5.8 | 0.8 | 1.2 | 3.8 | 11.1 | 1.6 | 2.3 | 7.2 |
| | | (13.8%) | (20.7) | (65.5%) | | (14.4%) | (20.7%) | (64.9%) |
| Testes | 3.3 | 0.4 | 0.9 | 2.0 | 6.4 | 0.9 | 1.7 | 3.8 |
| | | (12.1%) | (27.3%) | (60.6%) | | (14.1%) | (26.6%) | (59.3%) |
| Intestine | 2.9 | 0.2 | 0.8 | 1.9 | 6.0 | 0.5 | 1.7 | 3.8 |
| | | (6.9%) | (27.6%) | (65.5%) | | (8.3%) | (28.3%) | (63.4%) |

*The doses in the table were calculated by basing on the simulations of dose rates received in the organs as determined by the MCNP application, and also on the irradiation times and powers.

3. The Therapeutic Efficacy and Biological effects of BNCT for Osteosarcoma

Changes in the Rats' Body Weights.

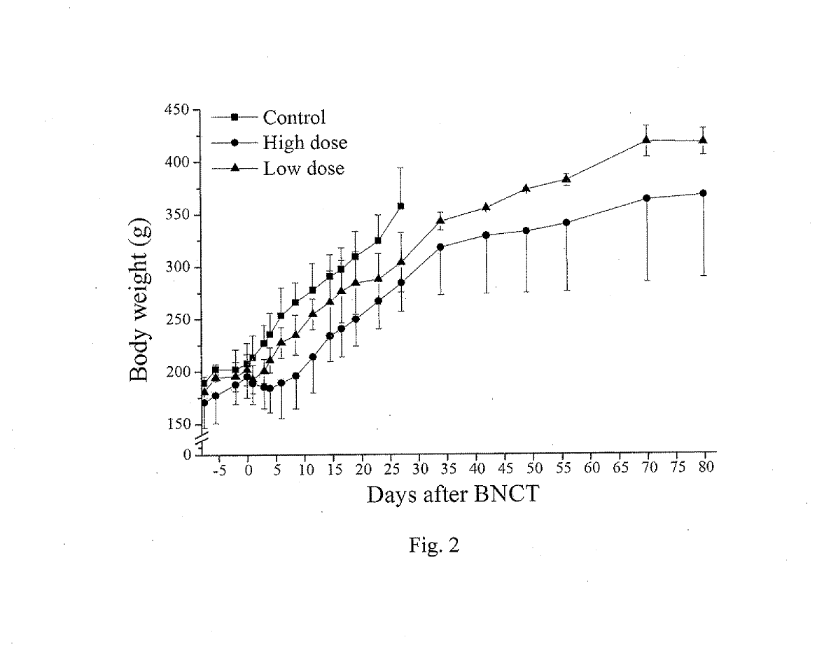

FIG. 2 shows changes in the body weight of the SD rats from the tumor control group, high-dose and low-dose BNCT groups after the BNCT treatment. The body weight of rat in each group was compared with that of the same rat on the day of BNCT treatment. On the 1st day after the irradiation, changes in the average body weight of the tumor control group, the high-dose and the low-dose BNCT groups were 2.9±0.5%, −3.3±1.0% and −4.5±2.2%; on the 3^{rd} day after the irradiation, changes in the average body weight of the three groups were 9.5±2.9%, −5.0±6.3% and −0.2±8.6%; on the 6^{th} day after the irradiation, changes in the average body weight of the three groups were 22.0±3.0%, −3.1±13.3% and 12.9±4.1%, and on the 8^{th} day after the irradiation, changes in the average body weight of the three groups were 28.5±6.9%, −0.4±13.4% and 16.5±8.3%. Comparing with the body weight on the day of the BNCT irradiation, the low-dose BNCT group of rats showed reductions in their body weights within three days of the BNCT treatment, but their body weights recovered after three days, while the high-dose BNCT group of rats showed continuous reductions in their body weights within one week after the BNCT treatment, though their body weights also recovered on the 8^{th} day after the treatment. After the irradiation, the rats initially showed symptoms of diarrhea and appetite loss, but the symptoms eased later, and the body weights recovered as well. The result suggested the gastro-intestinal reactions, weight loss, and other discomfort shown in the rats after the irradiation were temporary side-effects from the BNCT treatment.

Radiographic Analysis

The analysis of the radiographs were done before and after the treatment at the HsinChu Mackay Memorial Hospital weekly, by using a digital mammogram machine (Lorad Selenia, Lorad/Hologic®), in which the tumors were scanned under auto-filter and fixed imaging conditions (24 kVp, 12 mAs). The radiographs obtained under the fixed imaging condition allow the soft tissues to be observed more easily, thus the graphs were used to analyze tumor sizes, while the radiographs obtained under the auto-filter imaging condition allow the calcified tissues to be easily observed, and so the graphs were used to determine changes in the bone tissues.

Changes in Tumor Size

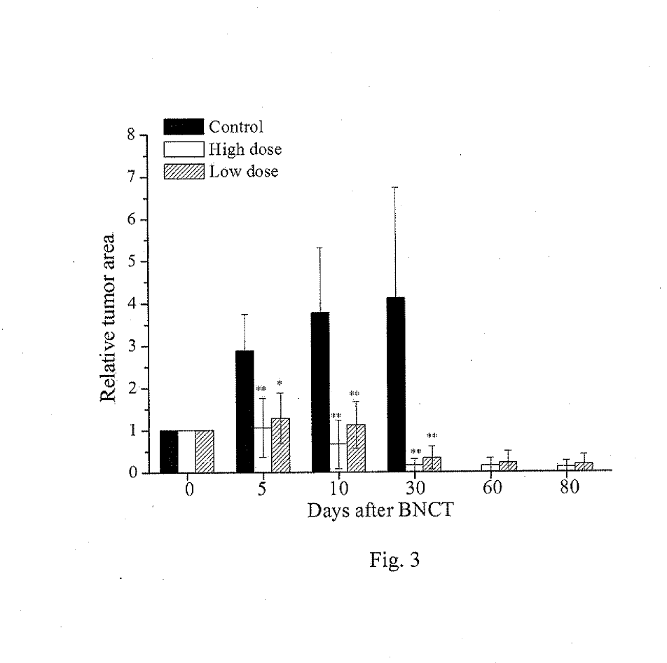

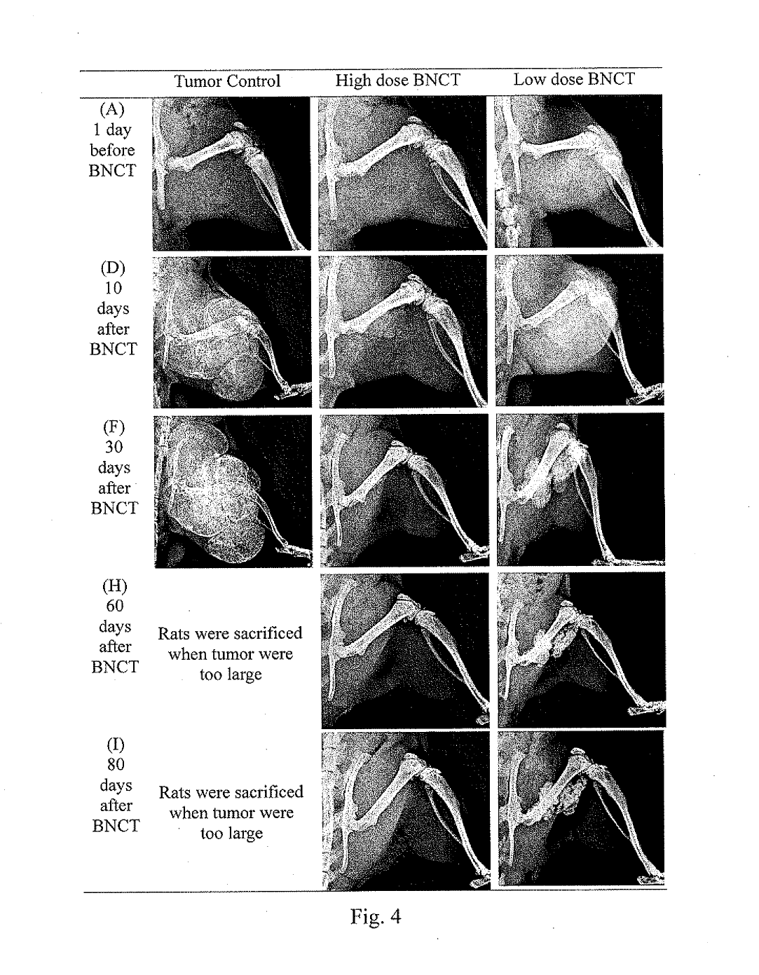

The image analysis software Image was used to select the scope of the extra-skeletal tumors, and then calculate tumor areas thereof to be used as an indicator of therapeutic efficacy. The relative tumor area of a SD rat was defined as a tumor area obtained from each time of imaging after the BNCT treatment divided by the tumor area obtained on the day of the BNCT treatment. FIG. 3 shows changes in the relative tumor area by tracing the tumor region after the osteosarcoma-bearing rats received BNCT treatment. On the 5^{th} day after the BNCT irradiation, the relative tumor areas of the tumor control group, the high-dose and the low-dose BNCT groups were 2.9, 1.1 and 1.3, respectively; on the 10^{th} day after the BNCT irradiation, the relative tumor areas of the three groups mentioned above were 3.8, 0.7 and 1.1, and on the 30^{th} day after the BNCT irradiation, the relative tumor areas of the three groups mentioned above were 4.1, 0.2 and 0.3. On the 30th day after the irradiation, the tumor size in the tumor control group of rats had grown to be excessively large (with tumor diameter >4 cm), thus the rats were euthanized. On the 60^{th} day after the irradiation, the relative tumor areas for the high-dose and the low-dose BNCT groups were both 0.2, and on the 80^{th} day after the irradiation, the relative tumor areas for the two groups were 0.1 and 0.2, respectively. On the 5th day after the treatment, the relative tumor area of the tumor control group was significantly higher than both the high-dose and the low-dose BNCT groups (p<0.01), and on the 30^{th} day after the BNCT treatment, the rate at which the tumors shrunk had slowed down, on the 60^{th} and 80^{th} days after the treatment, the tumor sizes had indeed reduced and showed no signs of recurrence. The radiographs indicated that: The smaller tumors found at the time of the treatment were vanished-after BNCT, while the larger tumors found at the time of the treatment were reduced in size, leaving scar tissues that displayed high density on the radiographs. FIG. 4 shows radiographs of the rats after receiving the BNCT treatment, it can be seen that the tumors continued to shrink after receiving the treatment, but the tumors without the treatment continued to grow larger.

Radiographs Show Bone Healing

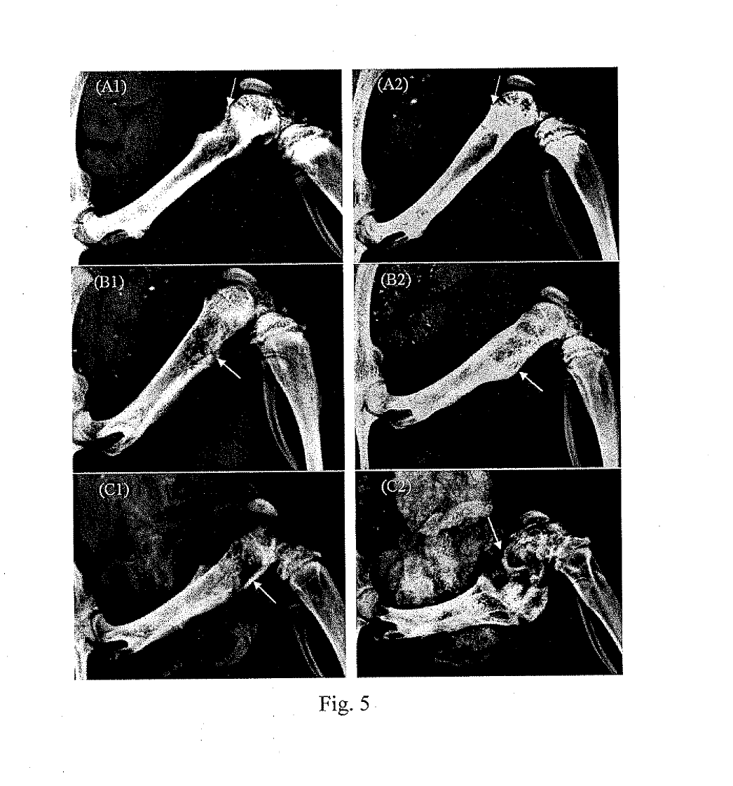

FIG. 5 show radiographs of the osteosarcoma-bearing rats with and without receiving BNCT treatment. FIG. 5-(A1) shows that on the 5^{th} day after the high-dose BNCT treatment, parts of the femurs showed visible radiolucent lines, as indicated by the arrows; FIG. 5-(B1) had arrows indicating the osteolytic lesion within cortical bone of distal posterior femur in the low-dose BNCT group. The observation suggests bone erosions continued shortly after the BNCT treatment. FIG. 5-(A2) shows the radiograph of the rats from FIG. 5-(A1) on the 16^{th} day after the treatment, in which the radiolucent line had healed, and the healed part displays increased radiodensity. FIG. 5-(B2) shows the radiograph of the rat from FIG. 5-(B1) on the 23rd day after the treatment, in which a continuous callus was seen passing through the fracture line, and the trabecular bone of the newly formed callus was tightly adhered to the surface of the cortical bone, and filled within the broken edges of the fracture. After the BNCT treatment, the bone appeared to be healing gradually.

FIG. 5-(C1) shows the femurs of the osteosarcoma-bearing rat without the BNCT treatment (the tumor control group). On the 16^{th} day after the tumor implantation (which was equal-to the 5^{th} day after BNCT of the treated groups), osteolytic lesion within cortical bone of distal posterior femur and elevation of periosteum could be observed; FIG. 5-(C2) shows the tumor control group on the 41st day after the tumor implantation (which was equal to the 30^{th} day after BNCT of the treated groups), in which severe full-thickness bicortical bone loss could be observed. The bone damage in the tumor control group of rats without the BNCT treatment had continued to deteriorate.

The Histopathological Examination of Tissues

### The Tumor and the Femur

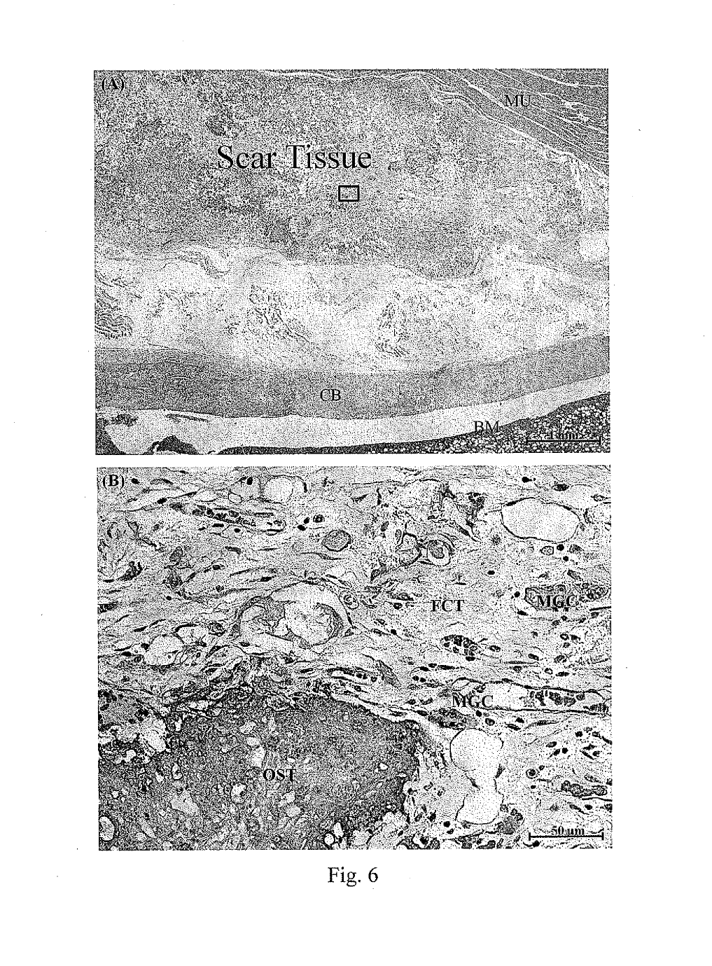

FIG. 6 shows the extra-skeletal tumor histopathological finding in osteosarcoma-bearing rats on the 80^{th} day after the BNCT treatment. FIG. 6-(A) are the histological section of the scar tissue resulting from the extra-skeletal regions of tumor after BNCT; FIG. 6-(B) shows the magnified view of the scar tissues boxed in FIG. 6-(A), which were fibrous connective tissues (FCT) and osteoids (OSD), wherein the dark grey areas (at bottom left of the figure) were osteoids, and the light grey areas were fibrous connective tissues and multinucleated giant cells (MGC); no-viable tumor cells were found in the sections.

The present invention provides a treatment of osteosarcoma by BNCT using boric acid as a single boron drug, which is effective for treating osteosarcoma. Neutron irradiation was carried out after administering boric acid into the rats. Subsequently, on the 5th day after the irradiation, the tumor areas between the rats that received BNCT treatment and those without had shown significant difference. On the 80^{th} day after the irradiation, the sizes of the tumors were reduced by 90% approximately, and the histopathological investigation-revealed the remaining tissues were osteoids and fibrous connective tissues, and no tumor cells were present. The present invention had provided proof showing boric acid could be used as a single boron drug for boron neutron capture therapy, and it is effective for treating osteosarcoma.

1. A method for treating osteosarcoma by boron neutron capture therapy (BNCT) comprising administering boric acid to a subject suffering osteosarcoma as a boron drug for BNCT, and subjecting the osteosarcoma in the subject to neutron irradiation.

2. The method according to claim 1, wherein the boric acid administered is an aqueous solution.

3. The method according to claim 2, wherein the administering comprises intravenously injecting the aqueous solution of boric acid to the subject.

4. The method according to claim 1, wherein the boric acid is constituted by boron having a major portion of ¹⁰B stable isotope.

5. The method according to claim 4, wherein the boric acid is constituted by boron having about 99% of ¹⁰B stable isotope.

6. The method according to claim 1, wherein said subject is human.

7. The method according to claim 4, wherein the boric acid is administered in an amount of 5-60 mg of ¹⁰B stable isotope per kilogram of human body weight.

8. The method according to claim 4 further comprising measuring a concentration of ¹⁰B stable isotope in the subject's blood, wherein said neutron irradiation is carried out according to the measured concentration.

9. The method according to claim 8, wherein said neutron irradiation is carried out when the measured concentration is higher than 20 μg ¹⁰B/g.

10. The method according to claim 3, wherein said neutron irradiation is carried out about 60-180 minutes after the intravenous injection.

11. The method according to claim 1, wherein said neutron irradiation is carried out with a physical dose of 5-15 Gy.

12. The method according to claim 11, wherein said neutron irradiation is carried out with a physical dose of 7-12 Gy.
